(19) United States
(12) Patent Application Publication
(10) Pub. No.: US 2024/0350830 A1
(43) Pub. Date: Oct. 24, 2024

(54) RADIOTHERAPY SYSTEM AND METHOD FOR CONTROLLING RADIOTHERAPY SYSTEM

(71) Applicants: KABUSHIKI KAISHA TOSHIBA, Tokyo (JP); TOSHIBA ENERGY SYSTEMS & SOLUTIONS CORPORATION, Kawasaki-shi (JP)

(72) Inventors: Yasuhiro SOEKAWA, Hachioji Tokyo (JP); Keiko OKAYA, Setagaya Tokyo (JP); Yoshifumi NAGAMOTO, Yokohama Kanagawa (JP); Fumi MARUYAMA, Hayama Kanagawa (JP)

(73) Assignees: KABUSHIKI KAISHA TOSHIBA, Toyko (JP); TOSHIBA ENERGY SYSTEMS & SOLUTIONS CORPORATION, Kawasaki-shi (JP)

(21) Appl. No.: 18/760,270

(22) Filed: Jul. 1, 2024

Related U.S. Application Data
(63) Continuation of application No. PCT/JP2023/000265, filed on Jan. 10, 2023.

(30) Foreign Application Priority Data
May 23, 2022 (JP) 2022-083785

(51) Int. Cl. A61N 5/10

(52) U.S. Cl. CPC A61N 5/107; A61N 5/1049; A61N 2005/1061

(57) ABSTRACT

According to one embodiment, a radiotherapy system comprising: a radioactive-ray irradiation apparatus configured to irradiate an irradiation target existing at an irradiation position with therapeutic radioactive rays; a 3D image acquisition apparatus configured to acquire a 3D image of the irradiation target at a position different from the irradiation position; a treatment table on which the irradiation target is placed; a treatment-table controller configured to control a position of the treatment table; and a memory configured to store the position of the treatment table for acquiring the 3D image as an imaging position.

### CROSS-REFERENCE TO RELATED APPLICATIONS

This application is a Continuation Application of No. PCT/JP2023/000265, filed on Jan. 10, 2023, and the PCT application is based upon and claims the benefit of priority from Japanese Patent Application No. 2022-083785, filed on May 23, 2022, the entire contents of which are incorporated herein by reference.

### TECHNICAL FIELD

Embodiments of the present invention relate to radiotherapy techniques.

### BACKGROUND

In positioning in radiotherapy, a CT image for treatment planning is compared with another CT image that is obtained by imaging a patient with a cone beam CT apparatus at an irradiation position of radioactive rays. Further, movement amount and rotation amount of the patient for accurately irradiating his/her lesion site with radioactive rays are calculated, i.e., movement amount and rotation amount of a treatment table on which the patient is fixed is calculated. In a particle beam therapy including a CT apparatus installed independently in a treatment room, CT imaging is usually performed in a state where the patient is placed near the irradiation position. At this time, there is a possibility of interference between the CT apparatus and an irradiation port. In order to avoid this interference, the irradiation port is configured to be movable in a known technique.

### PRIOR ART DOCUMENT

### Patent Document

[Patent Document 1] JP 2008-119380 A

[Patent Document 2] JP 2016-129639 A

### SUMMARY

### Problem to be Solved by Invention

When CT imaging is performed at a position different from the irradiation position, it is necessary to move the treatment table every time CT imaging is performed, and there is a problem in that it takes time to adjust the position of the treatment table. For example, when CT imaging is performed at a position different from the irradiation position during a planning stage for treatment and then CT imaging is performed again on the day of treatment or during treatment at a later date, the position of the treatment table needs to be adjusted all over again, which is time-consuming.

In view of the above-described circumstances, embodiments of the present invention aim to provide a radiotherapy technique that can facilitate movement of the treatment table at the time of acquiring a 3D (three-dimensional) image.

### BRIEF DESCRIPTION OF DRAWINGS

FIG. 1 is an overall configuration diagram illustrating a radiotherapy system according to the first embodiment.

FIG. 2 is a flowchart illustrating a radiotherapy procedure in the first embodiment.

FIG. 3 is a schematic diagram illustrating a flow of exchanging position/angle data in the first embodiment.

FIG. 4 is a flowchart illustrating a radiotherapy procedure in the second embodiment.

FIG. 5 is a flowchart illustrating a radiotherapy procedure in the third embodiment.

### DETAILED DESCRIPTION

In one embodiment of the present invention, a radiotherapy system comprising: a radioactive-ray irradiation apparatus configured to irradiate an irradiation target existing at an irradiation position with therapeutic radioactive rays; a 3D image acquisition apparatus configured to acquire a 3D image of the irradiation target at a position different from the irradiation position; a treatment table on which the irradiation target is placed; a treatment-table controller configured to control a position of the treatment table; and a memory configured to store the position of the treatment table for acquiring the 3D image as an imaging position.

According to embodiments of the present invention, it is possible to provide a radiotherapy technique that can facilitate movement of the treatment table at the time of acquiring a 3D (three-dimensional) image.

### First Embodiment

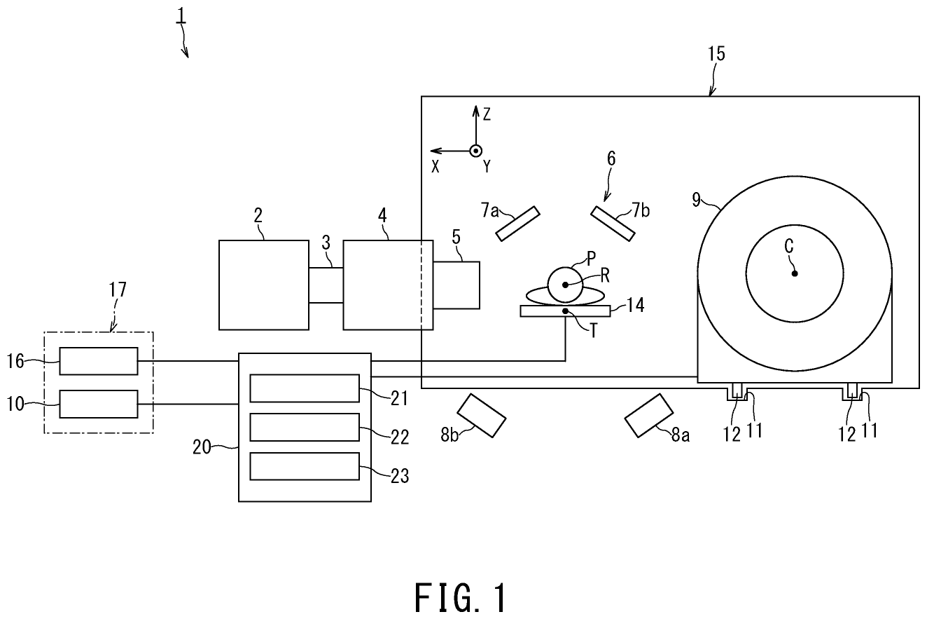

Hereinbelow, embodiments of a radiotherapy system and a method for controlling the radiotherapy system will be described in detail by referring to the accompanying drawings. The first embodiment will be described by using FIG. 1 to FIG. 3.

The reference sign 1 in FIG. 1 denotes the radiotherapy system according to the first embodiment. This radiotherapy system 1 is a so-called particle-beam cancer treatment apparatus that performs treatment by irradiating a diseased tissue such as a cancer tissue of a patient P with a particle beam such as carbon ions.

A radiation therapy with the use of the radiotherapy system 1 is also referred to as a heavy ion beam cancer treatment. This treatment is said to be able to damage the cancerous lesion (i.e., focus of disease) and minimize the damage to normal cells by pinpointing the cancerous lesion with carbon ions. Note that the particle beam is defined as radioactive rays heavier than an electron, and include a proton beam and a heavy ion beam, for example. Of this, the heavy ion beam is defined as radioactive rays heavier than a helium atom.

As compared with the conventional cancer treatment using X-rays, gamma rays, or proton beams, the cancer treatment using the heavy ion beam has characteristics that: (i) the ability to kill the cancerous lesion is higher; and (ii) the radiation dose is weak on the surface of the body of the patient so as to peak at the cancerous lesion. Thus, the number of irradiations and side effects can be reduced, and the treatment period can be shortened.

The particle beam loses its kinetic energy at the time of passing through the body of the patient P so as to decrease its velocity and receive a resistance that is approximately inversely proportional to the square of the velocity and stops rapidly when it decreases to a certain velocity. The stopping point of the particle beam is referred to as the Bragg peak at which high energy is emitted. The radiotherapy system 1 matches this Bragg peak with the position of the lesion tissue (i.e., affected part) of the patient, and thus, can kill only the lesion tissue while suppressing the damage to normal tissues.

The radiotherapy system 1 includes a particle beam generator 2, a transport path 3, a particle beam irradiation apparatus 4, and an X-ray imaging apparatus 6. Note that the particle beam irradiation apparatus 4 constitutes a radioactive-ray irradiation apparatus in the first embodiment.

The particle beam generator 2 generates a particle beam as therapeutic radioactive rays. The particle beam generator 2 generates a particle beam that is compatible with requirements of a treatment plan in terms of radiation type, energy, and dose. The transport path 3 transports the particle beam generated by the particle beam generator 2 to the particle beam irradiation apparatus 4.

The particle beam irradiation apparatus 4 includes an irradiation port 5 configured to radiate the transported particle beam onto a lesion site of the patient P, which is the irradiation target. The particle beam irradiation apparatus 4 controls the particle beam generated by the particle beam generator 2 in terms of irradiation position and irradiation timing so as to radiate the particle beam from the irradiation port 5 toward the patient P. Note that the irradiation port 5 is located inside a treatment room 15 where the patient P is to be treated.

The X-ray imaging apparatus 6 images the patient P by using X-rays (i.e., radioactive rays for imaging) in order to perform positioning of the patient P during radiotherapy in which the patient P is irradiated with the particle beam. The X-ray imaging apparatus 6 is disposed inside the treatment room 15.

The X-ray imaging apparatus 6 includes: two planar X-ray detectors 7a and 7b that are installed inside the treatment room 15 so as to be above the patient P placed at the irradiation position R; and two X-ray tubes 8a and 8b installed under the floor of the treatment room 15. The X-ray imaging apparatus 6 is configured to be able to acquire X-ray images of the patient P from two directions.

The radiotherapy system 1 further includes a CT apparatus 9 that performs computed tomography (hereinafter abbreviated as CT). Note that the CT apparatus 9 constitutes a 3D image acquisition apparatus in the first embodiment. The CT apparatus 9 images the patient P at a position C different from the irradiation position R so as to acquire a three-dimensional CT image of the patient P.

This CT apparatus 9 acquires a CT image of the patient P during radiotherapy, and is installed inside the treatment room 15 separately from the particle beam irradiation apparatus 4 and the X-ray imaging apparatus 6.

The CT apparatus 9 is self-propelled and is configured to be movable along two rails 11 laid on the floor surface of the treatment room 15. Specifically, the CT apparatus 9 includes a plurality of running wheels 12 that run on the two rails 11. One of these running wheels 12 is connected to a drive motor (not shown). When this drive motor is driven, the running wheels 12 run along the two rails 11. For example, when the vertical direction is defined as the Z-axis direction and a plane along the horizontal direction in the treatment room 15 is defined as a plane in parallel with both the X-axis direction and the Y-axis direction, the CT apparatus 9 can move only in the Y-axis direction of the treatment room 15.

The radiotherapy system 1 further includes a treatment table 14. The treatment table 14 is provided inside the treatment room 15. The treatment table 14 is a table on which the patient P is placed as the irradiation target during radiotherapy. Since the irradiation port 5 is fixed in the treatment room 15, positioning of the patient P is performed by moving the treatment table 14.

For example, the treatment table 14 can be moved in any of the X-axis direction, the Y-axis direction, and the Z-axis directions of the treatment room 15. In addition, the treatment table 14 can be tilted slightly to make fine adjustment to its position (angle). For example, the treatment table 14 can be rotated around the X-axis, the Y-axis, and the Z-axis.

The radiotherapy system 1 further includes: a pre-CT apparatus 10 that is for treatment planning and provided as a pre-image acquisition apparatus for the treatment planning and acquires CT images of the patient P during the planning stage for treatment; and a pre-treatment table 16 for the treatment planning and for placing the patient P thereon. The pre-CT apparatus 10 and the pre-treatment table 16 are provided inside a planning room 17, which is a room different from the treatment room 15.

The radiotherapy system 1 further includes a control computer 20. The treatment table 14, the pre-treatment table 16, the CT apparatus 9, and the pre-CT apparatus 10 are connected to the control computer 20 and are controlled by the control computer 20.

The control computer 20 includes a treatment-table controller 21, processing circuitry 22, and a memory 23. The control computer 20 includes hardware resources such as a Central Processing Unit (CPU), a Graphics Processing Unit (GPU), a Read Only Memory (ROM), a Random Access Memory (RAM), a Hard Disk Drive (HDD) and/or a Solid State Drive (SSD), and is configured as a computer in which information processing by software is achieved with the use of the hardware resources by causing the CPU to execute various programs. Further, the method for controlling the radiotherapy system 1 in the present embodiment is achieved by causing the computer to execute the various programs. For example, the treatment-table controller 21 and the processing circuitry 22 are implemented by causing its the CPU to execute the programs stored in the memory or the HDD.

Each component of the control computer 20 does not necessarily need to be provided in one computer. For example, one system may be realized by a plurality of computers interconnected via a network. For example, the treatment-table controller 21 and the memory 23 may be installed in individual computers. Furthermore, the treatment-table controller 21 and the processing circuitry 22 may be installed in individual computers.

The treatment-table controller 21 controls the position T of the treatment table 14. Note that the term “position” of the treatment table 14 in the present embodiment includes the meaning of the coordinates and angle (tilt) of the treatment table 14. In other words, the treatment-table controller 21 controls the movement amount and rotation amount at the time of operating the treatment table 14. Furthermore, the “position” of the treatment table 14 may be synonymous with the position of the patient P. The treatment-table controller 21 also controls the position of the pre-treatment table 16.

The memory 23 stores the position T of the treatment table 14 for acquiring a CT image as an imaging position. The imaging position to be stored in the memory 23 may be the position at which the CT image is acquired or may be the position at which the CT image is scheduled to be acquired. For example, aspects of causing the memory 23 to store the imaging position include: an aspect in which a future imaging position is stored without actually moving the treatment table 14; and another aspect in which a corrected imaging position is stored.

In addition, the treatment-table controller 21 moves the treatment table 14 on the basis of the imaging position stored in the memory 23. In this manner, a radiological technologist does not have to re-adjust the position T of the treatment table 14 from the beginning.

For example, the position at which the patient P is irradiated with the particle beam is defined as the irradiation position R. The irradiation position R is defined as fixed coordinates in three-dimensional space (i.e., in the treatment room 15). In general, the isocenter is set as the irradiation position R.

The X-ray imaging apparatus 6 generates X-ray images of the patient P at the irradiation position R. Note that the CT apparatus 9 generates a CT image by imaging the patient P at an imaging position different from the irradiation position R in order to avoid interference with the irradiation port 5. This imaging position is defined as coordinates in a three-dimensional space, and is set every time CT imaging is performed by the CT apparatus 9.

The treatment table 14 with the patient P placed thereon moves and rotates in the interior space of the treatment room 15 on the basis of instructions from the treatment-table controller 21.

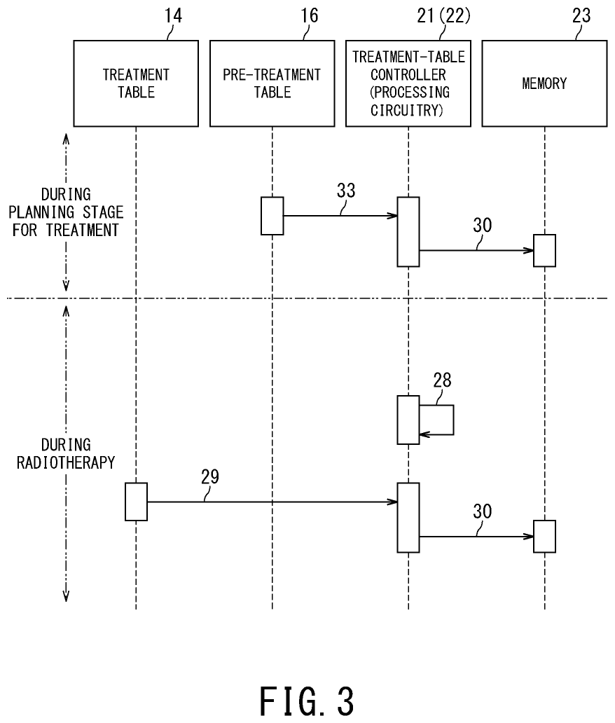

The position T of the treatment table 14 is expressed by the X-coordinate, Y-coordinate, and Z-coordinate indicating the position of the representative point of the treatment table 14 in the three-dimensional space inside the treatment room 15. This representative point may be the point on the flat plate on which the patient P is placed in the treatment table 14, for example. The angle (tilt) of the treatment table 14 is expressed by ψ, φ, and θ indicative of the rotation amount around the X-axis, the Y-axis, and the Z-axis. The six-dimensional quantities (X, Y, Z, ψ, φ, θ) including the position and angle of the treatment table 14 are referred to as position/angle data 29 for the treatment table 14 (FIG. 3). When the treatment table 14 operates, the treatment table 14 transmits the position/angle data 29 at this operation timing to the treatment-table controller 21.

The treatment-table controller 21 outputs instructions of movement and rotation to the treatment table 14. These instructions include: an instruction on the position and angle of the treatment table 14 as a movement/rotation destination; an instruction on movement amount/rotation amount of the treatment table 14; an instruction on a direction of movement/rotation of the treatment table 14; and an instruction of start and end of movement/rotation, for example. The position and angle of the treatment table 14 to be instructed by the treatment-table controller 21 are six-dimensional quantities (X, Y, Z, ψ, φ, θ). The movement amount/rotation amount of the treatment table 14 is the difference (ΔX, ΔY, ΔZ, Δψ, Δφ, Δθ) in position/angle of the treatment table 14. The direction of movement is a three-dimensional direction that includes at least one component of the X-axis direction, the Y-axis direction, and the Z-axis direction. The rotation direction is a three-dimensional direction that includes at least one component of the ψ direction, the φ direction, and the θ direction.

The processing circuitry 22 performs various calculations that are necessary for controlling the treatment table 14, the pre-treatment table 16, the CT apparatus 9, and the pre-CT apparatus 10.

For example, the processing circuitry 22 preliminarily or previously calculates a probability or possibility of mutual interference in the case of moving at least one of the CT apparatus 9 and the treatment table 14. Subsequently, when the processing circuitry 22 obtains a calculation result indicating that the mutual interference does not occur, the memory 23 stores the imaging position where this mutual interference does not occur. This configuration enables preliminary or advance checking as to whether the CT apparatus 9 and the treatment table 14 contact each other during movement or not. The processing circuitry 22 may also be configured to check whether the treatment table 14 comes into contact with any object other than the CT apparatus 9 or not.

During radiotherapy for the patient P in the treatment room 15 as shown in FIG. 3, before actual movement of the treatment table 14 or the CT apparatus 9, the processing circuitry 22 calculates whether the treatment table 14 or the CT apparatus 9 interferes with another object such as a predetermined device and a wall inside the treatment room 15 or not. This calculation is referred to as collision detection processing 28.

The treatment-table controller 21 includes a predetermined user interface. For example, when the radiological technologist operates a collision detection button on the user interface, the processing circuitry 22 performs the collision detection processing 28. Note that the collision detection processing 28 may be automatically executed at the same time as a user's operation for moving the treatment table 14 or the CT apparatus 9.

For example, when the radiological technologist operates a CT imaging-position storage button on the user interface, the treatment-table controller 21 receives the position/angle data 29 from the treatment table 14. The treatment-table controller 21 generates position/angle data 30 for CT imaging on the basis of both the received position/angle data 29 and the calculation result of the collision detection processing 28. The position/angle data 30 are stored in the memory 23.

When the processing circuitry 22 does not perform the collision detection processing 28, the received position/angle data 29 are stored as the position/angle data 30 in the memory 23.

The position/angle data 30 can be set as indicated values for the movement/rotation destination of the treatment table 14. Normally, in particle beam therapy, predetermined treatments are repeatedly performed on the same patient P in a predetermined order over a period of several days to several weeks. The treatment-table controller 21 can store the position/angle data 30 for each patient P, and the stored position/angle data 30 for each patient P can be read out or recalled even during the treatment on another day.

The pre-CT apparatus 10 generates a CT image for the treatment planning, and the patient P is placed on the pre-treatment table 16 at the time of CT-imaging for the treatment planning. In addition, the pre-treatment table 16 transmits position/angle data 33 of the treatment table to be used for the treatment planning to the treatment-table controller 21.

When any position/angle data 30 are not stored in the memory 23, the treatment-table controller 21 sets default values as the position/angle data 30. During imaging for generating a CT image to be used for the treatment planning, the treatment-table controller 21 receives the position/angle data 33 from the pre-treatment table 16. On the basis of the received position/angle data 33, the default values are updated and the position/angle data 30 are stored in the memory 23.

When it is determined from the calculation result of the collision detection processing 28 that collision does not occur, the treatment-table controller 21 stores the position/angle data 29 received from the treatment table 14 as the position/angle data 30 in the memory 23. This configuration eliminates the need to perform operations to store the position/angle data 30, and thus, can simplify user operations and the user interface.

In the first embodiment, during the planning stage for treatment, the position of the pre-treatment table 16 at the acquisition time of the CT image by the pre-CT apparatus 10 is stored as the imaging position in the memory 23. In this manner, on the basis of the positional relationship between the pre-CT apparatus 10 and the pre-treatment table 16 stored during the planning stage for treatment, the positional relationship between the CT apparatus 9 and the treatment table 14 can be reproduced during radiotherapy.

Furthermore, in the first embodiment, the CT apparatus 9 is movable. The memory 23 stores the position C of the CT apparatus 9 at the acquisition time of the CT image as an installation position. In this manner, the positional relationship between the CT apparatus 9 and the treatment table 14 at the previous acquisition time of the CT image can be reproduced at the time of acquiring the subsequent CT image.

Note that the imaging position indicates the relative positional relationship between the position T of the treatment table 14 and the position C of the CT apparatus 9. This imaging position does not have to be defined as fixed coordinates in the three-dimensional space (i.e., in the treatment room 15) but may be defined as the difference between the position T of the treatment table 14 and the position C of the CT apparatus 9, for example. In addition, the imaging position may be coordinates indicating the position T of the treatment table 14 under the definition that the origin is the position C of the CT apparatus 9. For example, when the CT apparatus 9 is moved, the position T of the treatment table 14, i.e., the imaging position is also moved accordingly.

Furthermore, at the time of acquiring the CT image, the CT apparatus 9 is moved on the basis of the installation position stored in the memory 23. In this manner, the CT apparatus 9 can be moved easily.

When one of both the imaging position of the treatment table 14 and the installation position of the CT apparatus 9 is stored in the memory 23, the processing circuitry 22 calculates the position of the other of both on the basis of the storage contents in the memory 23. The memory 23 stores the position of the other of both obtained by the processing circuitry 22. In this manner, when either the imaging position of the treatment table 14 or the installation position of the CT apparatus 9 can be obtained, the position of the other can also be obtained by calculation.

For example, the treatment-table controller 21 receives the installation position from the CT apparatus 9 and stores the installation position in the memory 23 as CT gantry position data (not shown). The CT-gantry position data are stored for each patient P, and can be read out or recalled for treatment on another day. In addition, the treatment-table controller 21 transmits the CT-gantry position data to the CT apparatus 9.

The CT apparatus 9 sets the CT-gantry position data (not shown) received from the treatment-table controller 21 as the installation position. In this configuration, the installation position at the time of the previous treatment can be reproduced during the second and subsequent treatments for the patient P.

The processing circuitry 22 may calculate the relative positional relationship from the CT-gantry position data (not shown) and the position/angle data 30 stored in the memory 23. In this configuration, during the second and subsequent treatments for the patient P, the relative positional relationship between the CT apparatus 9 and the treatment table 14 during the previous treatment can be reproduced on the basis of the installation position having been set, and thus, user operations are facilitated. Furthermore, on the basis of the position of the treatment table 14, the relative positional relationship between the CT apparatus 9 and the treatment table 14 at the time of the previous treatment can be reproduced, and consequently, user operations are facilitated.

Since the position/angle data 30 of the treatment table 14 for CT imaging are stored and the treatment table 14 is moved on the basis of these data on the day of treatment or during treatment at a later date in the above-described manner, movement of the treatment table 14 at the time of CT imaging can be easily performed.

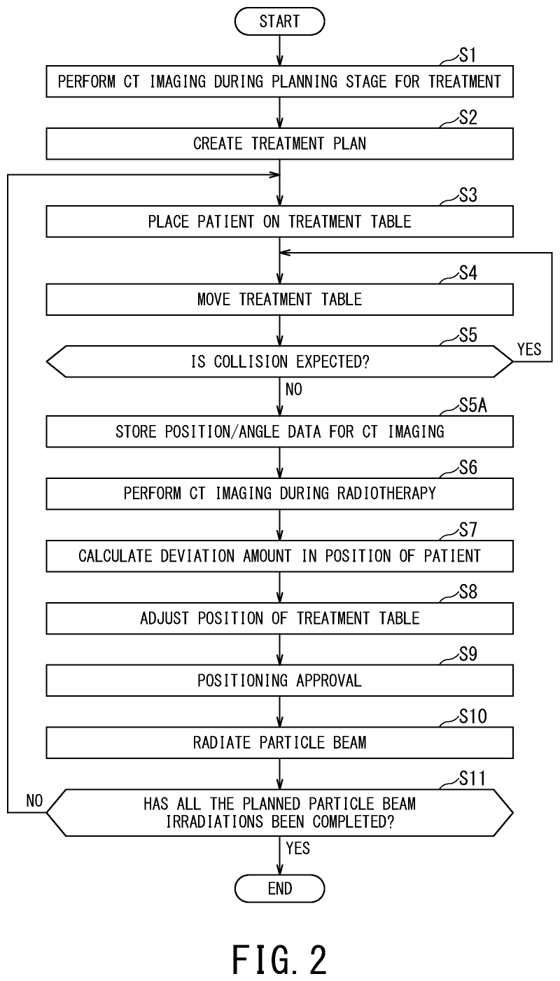

Next, a description will be given of the radiotherapy procedure in the first embodiment on the basis of the flowchart of FIG. 2 by referring to the above-described figured as required. The following steps are at least part of the processing included in the radiotherapy procedure, and the radiotherapy procedure may also include other processing.

In the first step S1, during the planning stage for treatment, the pre-CT apparatus 10 generate a CT image by imaging the patient P (FIG. 1). At this time, the radiological technologist marks the reference position at the time of imaging on the surface of a fixture (not shown) to be used for fixing the patient P on the pre-treatment table 16. The position/angle data 33 at the time of this imaging is transmitted to the treatment-table controller 21 (FIG. 3).

In the next step S2, a staff member such as a doctor and the radiological technologist creates a treatment plan by using the CT image acquired during the planning stage for treatment. In this treatment plan, the irradiation region of the particle beam is set. Furthermore, irradiation data are generated so as to include information on how to irradiate this irradiation region. The particle beam irradiation may be planned such that the total dose is divided into a plurality doses and the irradiation is repeated over several days to several weeks.

Next, the flow on the day of treatment will be described. In the step S3, the radiological technologist places the patient P on the treatment table 14 and fixes the patient P on the treatment table 14 by using a predetermined fixture (not shown).

In the next step S4, the radiological technologist adjusts the treatment table 14 in such a manner that the angle of the treatment table 14 becomes the same as in the treatment plan. For example, the treatment table 14 is translated (i.e., makes a translational movement) in such a manner that the position of the marking on the fixture matches the position at the time of CT imaging. Specifically, there is a reference laser irradiation point, and the marking position is aligned with this reference irradiation point. The treatment-table controller 21 sets the position/angle data 30 (FIG. 3) as the indicated values for the movement/rotation destination of the treatment table 14, and instructs the movement/rotation of the treatment table 14.

For example, in the case of the first treatment for the patient P, the position/angle data 30 (FIG. 3) reflect the angle of the pre-treatment table 16 at the time of CT imaging for the treatment planning. Thus, there is no need to adjust the angle of the treatment table 14, and only the translational movement of the treatment table 14 is performed. In this manner, operational errors at the time of adjusting the angle of the treatment table 14 can be prevented.

If the processing proceeds to the step of CT imaging in radiotherapy under the state where the treatment table 14 is at the wrong angle, CT imaging is performed again starting from the above-described step S4. For this reason, only the translational movement of the treatment table 14 is performed, and thus, the patient P is prevented from being exposed to extra-radiation due to re-execution of CT imaging.

In the case of the second and subsequent treatments, the position/angle data 30 (FIG. 3) are the data stored at the time of the previous treatment. Hence, there is no need to adjust the position and angle of the treatment table 14 after this. In this manner, the radiological technologist can move the treatment table 14 with simple operations.

In the next step S5, the processing circuitry 22 executes the collision detection processing 28 and determines whether the treatment table 14 is likely to collide with another object or not. If there is a possibility of collision (YES in the step S5), the processing returns to the step S4 and the position and angle of the treatment table 14 are adjusted in such a manner that any possibility of collision is excluded in the determination. If there is no possibility of collision (NO in the step S5), the processing proceeds to the step S5A.

In the step S5A, the radiological technologist operates the CT imaging-position storage button on the user interface. The treatment-table controller 21 stores the position/angle data 29 at this time in the memory 23 as the position/angle data 30 (FIG. 3).

In the next step S6, the control computer 20 drives the drive motor (not shown) of the CT apparatus 9. In response to this, the running wheels 12 run along the two rails 11, and thereby, the CT apparatus 9 moves to the imaging position. In this state, the annular gantry of the CT apparatus 9 is set in the region where the lesion site exists, and CT imaging is performed. After this CT imaging, the CT apparatus 9 is evacuated to the position before the CT imaging, as in the state before CT imaging.

In the next step S7, the processing circuitry 22 compares the CT image obtained through this imaging with the CT image previously obtained inside the treatment room 15, and calculates the deviation amount in the position of the lesion site. The treatment-table controller 21 sets this deviation amount and the difference between the imaging position and the irradiation position R to the indicated values of the movement amount/rotation amount of the treatment table 14.

In the next step S8, the treatment-table controller 21 performs positioning of the patient P by outputting a movement/rotation instruction to the treatment table 14. At this time, the treatment table 14 is located near the irradiation position R.

In the next step S9, the X-ray imaging apparatus 6 generates X-ray images of the patient P by imaging the patient P. After checking the position of the patient P, the radiological technologist approves the positioning. This positioning approval is performed by pressing a positioning approval button on the user interface, for example. When the positioning is approved, the position/angle data 29 at that time are transmitted from the treatment table 14 to the treatment-table controller 21 (FIG. 3). The treatment-table controller 21 then stores the received position/angle data 29 in the memory 23 as the position/angle data 30 for which positioning has been approved.

In the next step S10, the particle beam irradiation apparatus 4 irradiates the patient P with the particle beam. Here, the patient P is irradiated with an appropriate amount of particle beam in accordance with the treatment plan.

In the next step S11, the radiological technologist determines whether all the planned particle beam irradiations have been completed or not. If all the particle beam irradiations are completed (YES in the step S11), radiotherapy is completed. Otherwise, i.e., if at least one of the particle beam irradiations is not completed (NO in the step S11), the processing returns to the step S3 and the processing from the steps S3 to S10 is repeated until completion. This repetition is performed over several days to several weeks in some cases.

Although imaging of generating the CT image for the treatment planning is performed in the planning room 17 in the first embodiment, another aspect may be adopted. For example, imaging of generating the CT image for the treatment planning may be performed by using the CT apparatus 9 in the treatment room 15. At this time, when the radiological technologist operates the user interface, the position/angle data 29 at the time of imaging of generating the CT image for the treatment planning are stored as the position/angle data 30 in the memory 23 (FIG. 3). This configuration eliminates the need for the translational movement of the treatment table 14 during the first treatment for the patient P in the above-described step S4, which facilitates user operations. In such a configuration, the pre-CT apparatus 10 and the pre-treatment table 16 are no longer required, which can simplify the system configuration.

Furthermore, imaging for generating the CT image of the patient P may be performed after the particle beam irradiation. Here, the treatment-table controller 21 sets the position/angle data 30 as the indicated values of the movement/rotation destination of the treatment table 14, and instructs movement/rotation of the treatment table 14. In this manner, CT imaging can be performed at the same position and angle of the treatment table 14 as the position and angle at the time of CT imaging before the particle beam irradiation. Thus, the movement of the treatment table 14 can be facilitated. The generated CT image may be used for the purpose of checking the treatment effect, for example.

The radiotherapy system 1 may further include a treatment management computer (not shown) configured to manage treatment information on the patient P. This treatment management computer is provided with a second memory (not shown), for example. Information on the treatment is stored in the second memory for each patient P.

The information on the treatment may be information conforming to a Digital Imaging and Communications in Medicine (DICOM) format, which is a standard data format in radiotherapy.

Further, the treatment management computer stores the position/angle data 30 received from the control computer 20. In addition, the treatment management computer can transmit the stored position/angle data 30 to the control computer 20. Note that the control computer 20 may transmit the position/angle data 30 to the treatment management computer without storing the position/angle data 30 in the memory 23.

### Second Embodiment

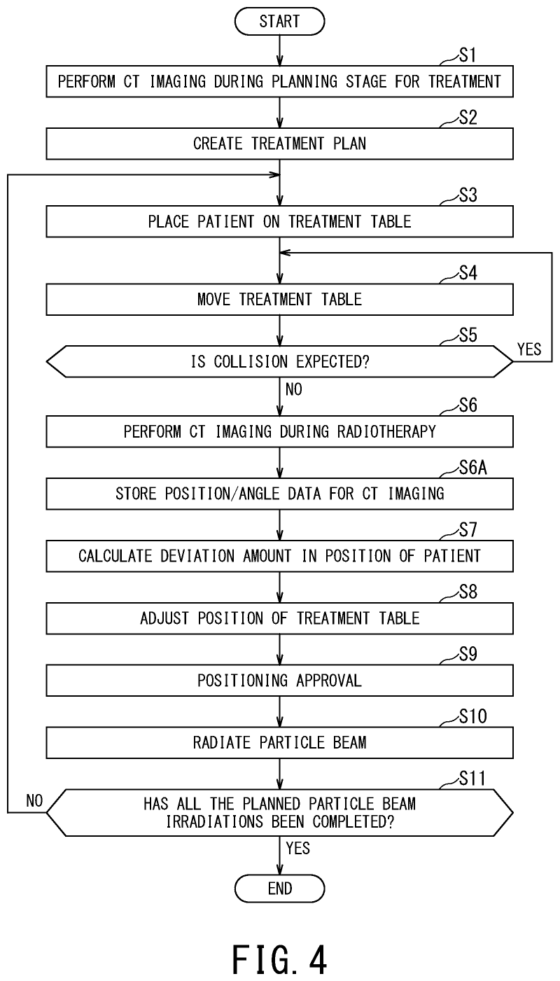

Next, the second embodiment will be described on the basis of FIG. 4 by referring to the above-described figures as required. The same components as those in the above-described embodiment are denoted by the same reference signs, and duplicate descriptions are omitted.

The radiotherapy procedure of the second embodiment will be described by using the flowchart of FIG. 4. The radiotherapy procedure in the second embodiment differs from the above-described first embodiment (FIG. 2) only in the timing at which the processing of storing the position/angle data 30 in the memory 23 is performed (step S6A), and is the same in the other steps as the first embodiment.

As shown in FIG. 4, in the step S6A subsequent to the step S6 (FIG. 3) in which CT imaging is performed, in response to this execution of CT imaging in the step S6, the treatment-table controller 21 stores the position/angle data 29 received from the treatment table 14 in the memory 23 as the position/angle data 30. In other words, the position/angle data 30 are updated with the position/angle data 29 received from the treatment table 14, and then the processing proceeds to the step S7.

The second embodiment eliminates the need for the radiological technologist to operate the CT imaging-position storage button on the user interface, and this configuration can simplify user operations and the user interface.

The memory 23 of the second embodiment stores the position of the treatment table 14 at the acquisition time of the CT image by the CT apparatus 9 as the imaging position (FIG. 1). In this configuration, the position of the treatment table 14 at the actual acquisition time of the CT image is stored, and thus, the previous position of the treatment table 14 can be reproduced at the next time of acquiring a CT image.

### Third Embodiment

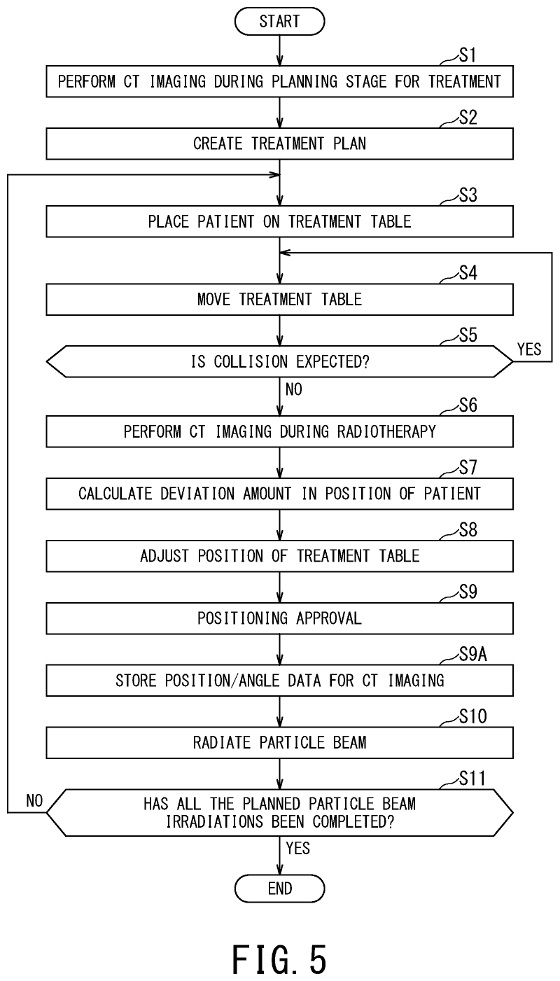

Next, the third embodiment will be described on the basis of FIG. 5 by referring to the above-described figures as required. The same components as those in the above-described embodiments are denoted by the same reference signs, and duplicate descriptions are omitted.

The radiotherapy procedure of the third embodiment will be described by using the flowchart of FIG. 5. The radiotherapy procedure in the third embodiment differs from the above-described first embodiment (FIG. 2) only in the timing at which the processing of storing the position/angle data 30 in the memory 23 is performed (step S9A), and is the same in the other steps as the first embodiment.

As shown in FIG. 5, in the step S9A subsequent to the step S9 (FIG. 3) in which the radiological technologist approves the positioning, in response to this positioning approval in the step S9, the treatment-table controller 21 stores the position/angle data 29 received from the treatment table 14 as the position/angle data 30 in the memory 23. In other words, the position/angle data 30 are updated with the position/angle data 29 received from the treatment table 14. This step S9A is automatically executed, and then, the processing proceeds to the step S10.

When the radiological technologist gives the positioning approval, the processing circuitry 22 performs a calculation of adding the difference between the irradiation position R and the imaging position to the position/angle data for the positioning approval (not shown). This calculation result is stored as the position/angle data 30 in the memory 23. Accordingly, this configuration eliminates the need for the radiological technologist to operate the CT imaging-position storage button on the user interface, and thus can simplify user operations and the user interface.

For example, in the step 7, the processing circuitry 22 calculates the movement amount and rotation amount of the treatment table 14 for matching the position T of the treatment table 14 with the irradiation position R at the time at which the particle beam irradiation apparatus 4 radiates the particle beam. In the step S9A, the calculation result obtained by the processing circuitry 22 is reflected in the imaging position stored in the memory 23.

In this manner, the position T of the treatment table 14 is finely adjusted at the time of the particle beam irradiation, and the movement amount and rotation amount of the treatment table 14 resulting from this fine adjustment can be reflected in the positioning of the treatment table 14 at the time of CT imaging to be performed by the CT apparatus 9. In addition, when the fine adjustment is performed at the time of CT imaging with the use of the CT apparatus 9, the movement amount and rotation amount of the treatment table 14 resulting from this fine adjustment can be reflected in the positioning of the treatment table 14 at the time of the particle beam irradiation.

As above, although the radiotherapy system 1 and its control method have been described on the basis of the first to third embodiments, the configuration applied in any one of the embodiments may be applied to other embodiments or the configurations in the respective embodiments may be applied in combination.

Although a mode in which each step is executed in series is illustrated in the flowcharts of the above-described embodiments, the execution order of the respective steps is not necessarily fixed and the execution order of part of the steps may be changed. Additionally, some steps may be executed in parallel with another step.

The control computer 20 in the above-described embodiments includes a storage device such as a Read Only Memory (ROM) and a Random Access Memory (RAM), an external storage device such as a Hard Disk Drive (HDD) and a Solid State Drive (SSD), a display device such as a display panel, an input device such as a mouse and a keyboard, a communication interface, and a control device which has a highly integrated processor such as a Central Processing Unit (CPU), a Graphics Processing Unit (GPU), a Field Programmable Gate Array (FPGA), and a special-purpose chip. The control computer 20 can be achieved by hardware configuration with the use of the normal computer.

Note that the program executed in the control computer 20 in the above-described embodiments is provided by being incorporated in a memory such as the ROM in advance. Additionally, or alternatively, the program may be provided by being stored as a file of installable or executable format in a non-transitory computer-readable storage medium such as a CD-ROM, a CD-R, a memory card, a DVD, and a flexible disk (FD).

In addition, the program executed in the control computer 20 may be stored on a computer connected to a network such as the Internet and be provided by being downloaded via a network. Further, the control computer 20 can also be configured by interconnecting and combining separate modules, which independently exhibit respective functions of the components, via a network or a dedicated line.

Although the patient P who is a human is illustrated as the irradiation target in the above-described embodiments, an animal such as a dog and a cat may be used as the irradiation target, and the radiotherapy system 1 may also be used at the time of performing particle beam therapy on such an animal.

Although the CT apparatus 9 is movable only in the Y-axis direction of the treatment room 15 in the above-described embodiments, another aspect may be adopted. For example, the CT apparatus 9 may be freely movable in any of the X-axis direction, the Y-axis direction, and the Z-axis direction of the treatment room 15. In addition, CT imaging may be performed at the irradiation position R.

Although the CT apparatus 9 is illustrated as the 3D image acquisition apparatus in the above-described embodiments, another aspect may be adopted. For example, a magnetic resonance image (MRI) apparatus may be used as the 3D image acquisition apparatus.

Although the particle beam is illustrated as therapeutic radioactive rays in the above-described embodiments, another aspect may be adopted. For example, the therapeutic radioactive rays may also be other radioactive rays such as X-rays, gamma rays, and proton beams.

According to at least one embodiment described above, the memory 23 configured to store the position T of the treatment table 14 for acquiring a 3D image as the imaging position is provided, and thus, movement of the treatment table 14 at the time of acquiring the 3D image can be readily performed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions. The articles “the”, “a” and “an” are not necessarily limited to mean only one, but rather are inclusive and open ended so as to include, optionally, multiple such elements.

1. A radiotherapy system comprising:
a radioactive-ray irradiation apparatus configured to irradiate an irradiation target existing at an irradiation position with therapeutic radioactive rays;
a 3D image acquisition apparatus configured to acquire a 3D image of the irradiation target at a position different from the irradiation position;
a treatment table on which the irradiation target is placed;
a treatment-table controller configured to control a position of the treatment table; and
a memory configured to store the position of the treatment table for acquiring the 3D image as an imaging position.

2. The radiotherapy system according to claim 1, wherein the treatment-table controller is configured to move the treatment table in accordance with the imaging position stored in the memory.

3. The radiotherapy system according to claim 1, further comprising processing circuitry configured to preliminarily calculate a possibility of mutual interference in a case of moving at least one of the 3D image acquisition apparatus and the treatment table,
wherein, when the processing circuitry obtains a calculation result indicating that the mutual interference does not occur, the memory stores the imaging position where the mutual interference does not occur.

4. The radiotherapy system according to claim 1, wherein the memory is configured to store, as the imaging position, the position of the treatment table at a time at which the 3D image is acquired by the 3D image acquisition apparatus.

5. The radiotherapy system according to claim 1, further comprising processing circuitry configured to calculate movement amount and rotation amount of the treatment table for aligning the position of the treatment table with the irradiation position when the radioactive-ray irradiation apparatus radiates the therapeutic radioactive rays,
wherein the memory is configured to reflect a calculation result obtained by the processing circuitry in a stored imaging position.

6. The radiotherapy system according to claim 1, further comprising:
a pre-image acquisition apparatus configured to acquire the 3D image of the irradiation target during a planning stage for treatment; and
a pre-treatment table on which the irradiation target is placed during the planning stage for treatment,
wherein the memory is configured to store, as the imaging position, a position of the pre-treatment table at a time at which the 3D image is acquired by the pre-image acquisition apparatus during the planning stage for treatment.

7. The radiotherapy system according to claim 1, wherein:
the 3D image acquisition apparatus is configured to be movable; and
the memory is configured to store, as an installation position, a position of the 3D image acquisition apparatus at a time at which the 3D image is acquired.

8. The radiotherapy system according to claim 7, wherein the 3D image acquisition apparatus is configured to perform movement in accordance with the installation position stored in the memory at a time of acquiring the 3D image.

9. The radiotherapy system according to claim 7, further comprising processing circuitry,
wherein, when one of both the imaging position of the treatment table and the installation position of the 3D image acquisition apparatus is stored in the memory, the processing circuitry calculates a position of another of the both by using storage contents in the memory and then stores the position of another of the both in the memory.

10. A method for controlling a radiotherapy system by using:
a radioactive-ray irradiation apparatus configured to irradiate an irradiation target existing at an irradiation position with therapeutic radioactive rays;
a 3D image acquisition apparatus configured to acquire a 3D image of the irradiation target at a position different from the irradiation position;
a treatment table on which the irradiation target is placed;
a treatment-table controller configured to control a position of the treatment table; and
a memory,
the method comprising a step of causing the memory to store the position of the treatment table for acquiring the 3D image as an imaging position.
